(19) United States
(12) Patent Application Publication
(10) Pub. No.: US 2009/0099780 A1
(43) Pub. Date: Apr. 16, 2009

(54) HEALTHCARE DEVICE AND COMPREHENSIVE MEASUREMENT METHOD USING THE SAME

(75) Inventors: Cheng-I Yang, Jhonghe (TW); Ping-Tse Lin, Jhonghe (TW); Jia-Sheng Heh, Jhonghe (TW); Weichih Hu, Pingjhen (TW)

Correspondence Address: HEALTHY BIOTECH CORP. LTD., P.O. BOX 3-161, BANCIAO, TAIPEI COUNTY, 22099, TW

(73) Assignee: HEALTHY BIOTECH CORP. LTD., Jhonghe (TW)

(21) Appl. No.: 11/872,709

(22) Filed: Oct. 16, 2007

(51) Int. Cl. G01N 33/48

(52) U.S. Cl. 702/19

(57) ABSTRACT

A healthcare device and a method using the same are disclosed. The healthcare device includes a biological data measuring module, a conversion module for amplifying data measured by the biological data measuring module by using a non linear function, a biological data input module for inputting a conversion data converted by said conversion module or for inputting a measuring data measured by a Chinese or a western medical device, a healthcare classification module for generating different healthcare classification data according to comparison of data inputted by the biological data input module, and a biological performance module for converting the classified healthcare clue into an energy distribution data or a mentality level data by using a non linear function.

### BACKGROUND OF THE INVENTION

1. Field of the Invention

The present invention generally relates to a healthcare device and a comprehensive measurement method using the same.

2. Description of Related Art

The general biological signal analysis system comprises a biological signal collector and a non-linear graphic analysis device. The biological signal collector generates a biological signal in response to measuring the living object, and the non-linear graphic analysis device analyzes the biological signal with the graphic feature and fractionates the graphic feature of the biological signal in order to measure and to analyze the living object.

The conventional biological signal collector of the biological signal analysis system has an amplifying circuit for amplifying the biological signal, which would inevitably increase the cost of the system. Furthermore, the system can only accept the biological signal generated by the biological signal collector rather than directly accepting the data of the biological signal. Therefore, there is a need to improve the conventional biological signal analysis system.

### SUMMARY OF THE INVENTION

Accordingly, the present invention provides a healthcare device and a comprehensive measurement method using the same to obtain a comprehensive biological data of the living object.

In accordance with the above objectives of the present invention, a healthcare device is provided, which comprises a measuring module for measuring a living object and generate a biological signal, and then the biological signal is converted into a biological data. Both Chinese and western medical devices can accept the biological data, and therefore the defect of the conventional biological signal analysis system described above may be overcome. The present invention uses a non-linear function to amplify the biological data obtained by converting the biological signal, and therefore the cost can be effectively reduced.

The healthcare device and the comprehensive measurement method of the present invention can also advantageous use the standard deviation probability scheme to classify the biological data to provide a reliable guideline to the healthcare classification. Furthermore, the data of the healthcare classification can be converted into energy distribution data or mentality level data.

### BRIEF DESCRIPTION OF THE DRAWING

For a more complete understanding of the present invention, reference will now be made to the following detailed description of preferred embodiments taken in conjunction with the following accompanying drawings.

FIG. 1A-1B shows flowcharts of a comprehensive measurement method according to an embodiment of the present invention.

FIG. 2 is a view of a healthcare device an embodiment of the present invention.

### DETAIL DESCRIPTION OF THE INVENTION

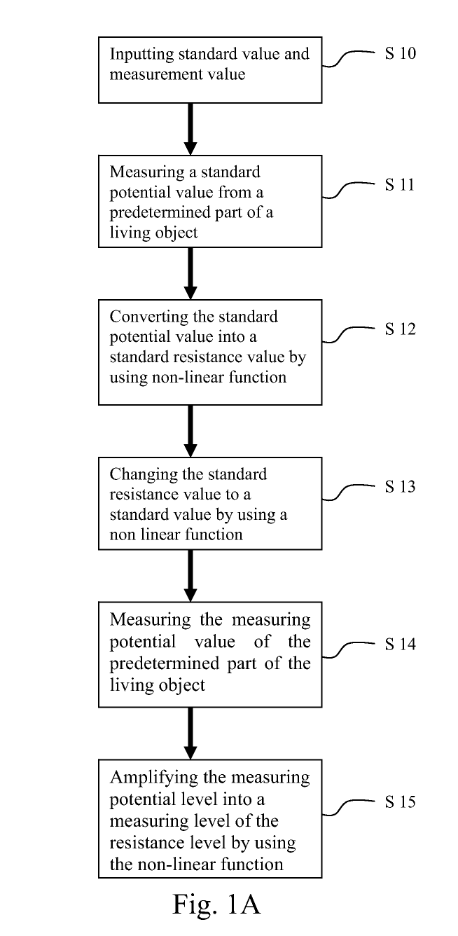

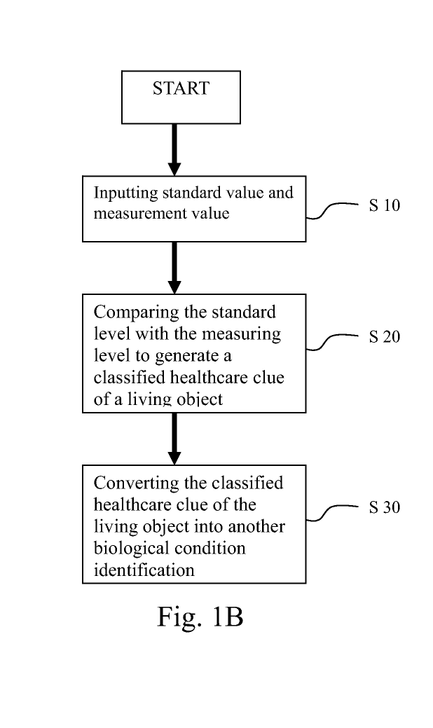

FIG. 1A-1B shows flowcharts of a comprehensive measurement method according to an embodiment of the present invention. Referring to FIG. 1A, first, at step S10, a set of corresponding standard value and measurement value of the biological signal measured by a Chinese or a western medical device is input or measured. Next, at step S11, a standard potential value of a potential value is measured from a predetermined part of a surface of a living object to generate a micro current signal. Next, at step S12, the standard potential value is converted into a standard resistance value to amplify the standard potential value and convert it into the standard resistance value by using the non-linear function (for example, index function). Next at step S113, the standard resistance value is changed to a standard value of a predetermined part of the living object by using the non-linear function. Next at step S14, the potential value of the predetermined part of the living object is measured. Next, at step S15, the potential value is amplified into a measured value of the resistance value by using the non-linear function (for example, index function).

Referring to FIG. 1B, at step S20, the standard value is compared with the measured value to generate a healthcare clue of a living object. The classified healthcare clue may be defined according as follows:
1. normal, when (standard value−standard deviation)<measured value≦standard value;
2. secondary normal, when (standard value−1.5*standard deviation)<measured value≦(standard value−standard deviation);
3. caution, when (standard value−2*standard deviation)<measured value≦(standard value−1.5*standard deviation);
4. attend, when (standard value−2*standard deviation)<measured value≦(standard value−1.5*standard deviation).

The radicand of the average of the square of difference from every measured value and the average measured value is a standard deviation; and the difference between the measured values may be achieved by measuring the probability distribution.

At step S30, the classified healthcare clue is converted into an additional biological condition. In other words, the additional biological condition includes, for example, energy distribution data or the mentality level data, which may be obtained by converting the data of the classified healthcare clue by the non linear function.

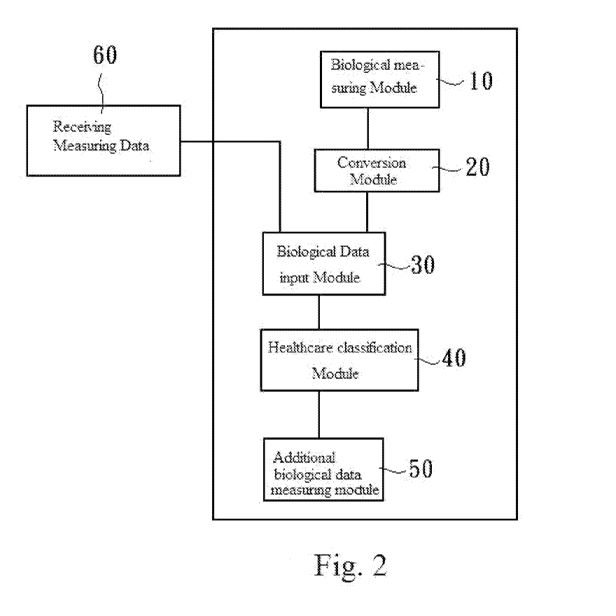

FIG. 2 is a view of a healthcare device according an embodiment of the present invention. Referring to FIG. 2, the healthcare device comprises a biological data measuring module 10, a conversion module 20, a biological data input module 30, a healthcare classification module 40 and an additional biological performance module 50.

The biological data input module 30 receives the measuring data 60 inputted by a Chinese/western medical device or a conversion data from the conversion module 20. The conversion data from the conversion module 20 is generated by the biological data measuring module 10 via a non-invaded method to output a micro current signal corresponding to a predetermined part of the living object, and the biological data measuring module 10 further measures the potential value of the signal, the potential value is then converted into a measured value to amplify and to convert it into a resistance value by the non-linear function (for example, the index function). The biological data input module 30 comprises the resistance value of a part of the living object and the standard resistance to enable the healthcare classification module 40 to compare these values with the standard deviation in the probability distribution in order to output a classified healthcare clue of the living object. The classified healthcare clue value is then converted into energy distribution data or mentality level data by using the non linear function via the additional biological performance module 50.

While the invention has been described in conjunction with a specific best mode, it is to be understood that many alternatives, modifications, and variations will be apparent to those skilled in the art in light of the foregoing description. Accordingly, it is intended to embrace all such alternatives, modifications, and variations in which fall within the spirit and scope of the included claims. All matters set forth herein or shown in the accompanying drawings are to be interpreted in an illustrative and non-limiting sense.

What is claimed is:

1. A comprehensive measurement method for a healthcare device, comprising:
(a) inputting at least one standard value and a measured value of a biological signal;
(b) comparing the standard value with the measured value to generate a classified healthcare clue of a living object; and
(c) converting the classified healthcare clue into at least one biological data.

2. The comprehensive measurement method for a healthcare device according to claim 1, wherein a data of the standard value and the measured value of the biological signal in step (a) is measured by using a Chinese or a western medical device, which comprises the following sub-steps:
(a1) measuring a standard potential value of a potential value from a predetermined part on a surface of a living object;
(a2) converting the standard potential value into a standard resistance value;
(a3) converting said standard resistance value into a standard value of a predetermined part of the living object;
(a4) measuring a potential value of a predetermined part on a surface of the living object; and
(a5) converting the potential value into a measured value.

3. The comprehensive measurement method for a healthcare device according to claim 1, wherein the classified healthcare clue includes:
(b1) normal when (standard value−standard deviation)<measured value≦standard value;
b2) secondary normal when (standard value−1.5*standard deviation)<measured value≦(standard value−standard deviation);
b3) caution when (standard value−2*standard deviation)<measured value≦(standard value−1.5*standard deviation); and
b4) attend when (standard value−2*standard deviation)<measured value≦(standard value−1.5*standard deviation).

4. The comprehensive measurement method for a healthcare device according to claim 1, wherein the step (c) is performed by using a non linear function.

5. The comprehensive measurement method for a healthcare device according to claim 1, wherein the biological data in step (c) includes an energy distribution data or a mentality level data.

6. A healthcare device, comprising
at least one biological data measuring module;
at least one conversion module, for amplifying data measured by the biological data measuring module by using a non linear function;
at least one biological data input module, for inputting a conversion data converted by the conversion module or for inputting a measuring data measured by a Chinese or a western medical device; and
at least one healthcare classification module, for generating different healthcare classification data according to comparison of data inputted by the biological data input module.

7. The healthcare device according to claim 6, further comprising a biological performance module for converting the classified healthcare clue into an energy distribution data or a mentality level data by using a non linear function.
